(19) United States
(12) Patent Application Publication
(10) Pub. No.: US 2015/0126862 A1
(43) Pub. Date: May 7, 2015

(54) Angiographic Examination Method for the Detection of Microembolizations

(71) Applicant: Marcus Pfister, Bubenreuth (DE)

(72) Inventor: Marcus Pfister, Bubenreuth (DE)

(21) Appl. No.: 14/535,115

(22) Filed: Nov. 6, 2014

(30) Foreign Application Priority Data
Nov. 7, 2013 (DE) 102013222674.8

(51) Int. Cl. A61B 6/00

(52) U.S. Cl. CPC A61B 6/504; A61B 6/481; A61B 6/5235
USPC 600/431

(57) ABSTRACT

An angiographic examination method for performing a subtraction angiography for the detection of microembolizations includes capturing at least one pre-operative pre-contrast image and at least one pre-operative post-contrast image having a vascular tree filled with contrast medium. The method also includes subtracting the pre-contrast image and the post-contrast image in order to produce a pre-operative subtraction image. The method includes capturing at least one post-operative post-contrast image having a vascular tree filled with contrast medium, and subtracting a pre-contrast image and the post-operative post-contrast image in order to produce a post-operative subtraction image. The method also includes subtracting the pre-operative subtraction image and the post-operative subtraction image in order to produce a third subtraction image, and reproducing the third subtraction image.

This application claims the benefit of DE 10 2013 222 674.8, filed on Nov. 7, 2013, which is hereby incorporated by reference in its entirety.

### BACKGROUND

The present embodiments relate to an angiographic examination method.

After heart attacks and cancer, strokes are the third most common cause of death. In addition, strokes are the most common cause for the need for care for the elderly. In Germany, some 200,000 people a year suffer a stroke, not including the unnoticed, “silent” cerebral infarctions. A high percentage of the stroke cases are caused by a constriction of the carotid artery and in the majority of cases, as a result of arteriosclerosis. If a higher-grade carotid stenosis is present, this is to be treated. This may take place either in open surgery (OP) or in endovascular fashion by widening the carotid artery using carotid stenting. In this situation, the stenosis is expanded by a balloon, and then, a stent that supports the vessel wall is inserted. The treatments are performed on angiography systems, such as, for example, the Siemens Artis Zee®. Such an angiography system is known, for example, from U.S. Pat. No. 7,500,784 B2, which is described with reference to FIG. 1.

FIG. 1 shows a monoplanar X-ray system, illustrated in an exemplary manner, with a C-arm 2 held by a stand 1 in the form of a six-axis industrial or folding-arm robot. Attached to ends of the six-axis industrial or folding-arm robot, there are an X-ray radiation source (e.g., an X-ray emitter 3 with X-ray tube and a collimator) and an X-ray image detector 4 as an image recording unit.

Using the folding-arm robot known, for example, from U.S. Pat. No. 7,500,784 B2, which may have six axes of rotation and therefore six degrees of freedom, the C-arm 2 may be adjusted spatially, as required (e.g., via the C-arm 2 being rotated about a center of rotation between the X-ray emitter 3 and the X-ray image detector 4). The angiographic X-ray system 1 to 4 according to one or more of the present embodiments is rotatable, for example, about centers of rotation and axes of rotation in the C-arm plane of the X-ray image detector 4 (e.g., about the center point of the X-ray image detector 4 and about axes of rotation intersecting the center point of the X-ray image detector 4).

The known folding-arm robot has a main frame that, for example, is securely mounted on a floor. A carousel is affixed thereto in a manner rotatable about a first axis of rotation. A robot rocker is attached pivotably about a second axis of rotation on the carousel. A robot arm is rotatably attached on the robot rocker about a third axis of rotation. A robot hand is attached rotatably about a fourth axis of rotation at the end of the robot arm. The robot hand has an attachment element for the C-arm 2, which may be pivoted about a fifth axis of rotation and may be rotated about a sixth axis of rotation extending perpendicular thereto.

The realization of the X-ray diagnostics apparatus is not dependent on the industrial robot. Conventional C-arm devices may also be used.

The X-ray image detector 4 may be a rectangular or quadratic, flat semiconductor detector that may be made from amorphous silicon (a-Si). However, integrating and possibly counting CMOS detectors may also be used.

A patient 6 to be examined as an examination object is situated on a table surface 5 of a patient mounting table in the beam path of the X-ray emitter 3. Attached to the X-ray diagnostics apparatus is a system control unit 7 with an imaging system 8 that receives and processes the image signals from the X-ray image detector 4 (operating elements, for example, are not illustrated). The X-ray images then may be viewed on displays of a suspended monitor bracket 10 supported by a ceiling-mounted carrier system 9 that may track longitudinally, may pivot, may rotate and is adjustable in height.

In place of the X-ray system with the stand 1 in the form of the six-axis industrial or folding-arm robot illustrated in FIG. 1 in an exemplary manner, the angiographic X-ray system may also have a normal ceiling-mounted or floor-mounted bracket for the C-arm 2.

In place of the C-arm 2, which is illustrated in an exemplary manner, the angiographic X-ray system may also have separate ceiling-mounted and/or floor-mounted brackets for the X-ray emitter 3 and the X-ray image detector 4 which, for example, are coupled in an electronically rigid manner.

In the case of vascular dilation by carotid stenting, plaques may become detached, which may then trigger embolisms in the brain (i.e., a stroke). In order to avoid this situation, the physician may introduce a filter that intercepts the detached pieces. Nevertheless, microembolizations (e.g., “hits”) are produced time and time again as a result of the procedures.

These hits may be diagnosed, for example, using digital subtraction angiography (DSA), the basic principle of which will be described briefly with reference to FIG. 2. In this situation, a plurality of chronologically sequential X-ray images of the brain or skull 11 are produced. At least one X-ray image of the plurality of chronologically sequential X-ray images of the brain or skull 11 is saved as a pre-contrast image or mask image 12 with no contrast medium. During the imaging sequence, the vessels are injected with contrast medium in the filling phase, which provides that further images having contrast medium distribution result. At least one of the further images is saved as a post-contrast image 13 with contrast medium, in which a vascular tree 14 filled with contrast medium may be recognized. The digital mask image 12 is subtracted in a subtraction 15 from the later images (e.g., the post-contrast images 13). What remains are only parts or regions of the image that differ from one another (e.g., precisely the blood vessels of the vascular tree 14 filled with contrast medium), as may be recognized in a subtraction image 16. The vascular tree 14 filled with contrast medium exhibits fine details or branching 17.

If the mask images 12 and post-contrast images 13 are not precisely registered, the outline 18 of the skull may, however, only be faintly recognized, as is intended to be indicated by the dotted reproduction thereof in the subtraction image 16.

Anatomical details not of interest are therefore “subtracted away” by the DSA such that only the blood vessels of interest alone are visible.

The diagnosis of a stroke may take place using visual inspection of the post-operative DSAs or subtraction images 16 by a physician or investigator. Since the occlusions are, however, very small in general, the occlusions may only be recognized with difficulty in these angiographs.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an angiographic examination method where microembolizations or “hits” may be better and more easily recognized is provided.

An angiographic examination method according to one or more of the present embodiments includes capturing at least one pre-operative pre-contrast image and at least one pre-operative post-contrast image having a vascular tree filled with contrast medium. The pre-contrast image and the post-contrast image are subtracted in order to produce a pre-operative subtraction image. At least one post-operative post-contrast image having a vascular tree filled is captured with contrast medium. A pre-contrast image and the post-operative post-contrast image are subtracted in order to produce a post-operative subtraction image. The pre-operative subtraction image and the post-operative subtraction image are subtracted in order to produce a third subtraction image, and the third subtraction image is reproduced.

This design of the angiographic examination method according to one or more of the present embodiments facilitates the location of the smallest microembolizations or “hits”, for example, following a carotid stenting. A better visualization of the microembolizations is achieved through the comparison of pre-operative and post-operative angiographs using DSA.

Variations caused by different fillings with contrast medium may be avoided according to one or more of the present embodiments if images of an entire sequence are added together in order to capture the at least one pre-operative pre-contrast image, the at least one pre-operative post-contrast image, and the at least one post-operative post-contrast image.

It may be advantageous if a motion compensation is performed during at least one of the subtractions.

According to one or more of the present embodiments, the images may be obtained from volume data from 3D rotational angiographs, which provides that 3D visualizations of the vascular system (e.g., 3D blood-flow visualizations or 3D perfusion visualizations) are reproduced.

Motion artifacts of DSA sequences with respect to one another may be avoided if at least one registration is performed prior to the subtractions.

The at least one registration may, for example, be a rigid or flexible 2D/2D registration in the case of DSA images or a 3D/3D registration in the case of volume data sets.

The “blocked” portions remaining in the subtraction image may be superimposed in color in one of the DSA or volume images or one of the DSA or volume sequences such that although “blocked” portions are clearly visible, the “blocked” portions do, however, remain in the anatomical context if, according to one or more of the present embodiments, the contents of the third subtraction image are overlaid in color-coded fashion onto the post-operative subtraction image.

In one embodiment, the pre-operative subtraction image and the post-operative subtraction image may be visualized in color-coded fashion and subtracted from one another in color-coded fashion.

According to one or more of the present embodiments, 4D DSA sequences may be subtracted from one another in order to perform at least one of the subtractions.

### BRIEF DESCRIPTION OF THE DRAWINGS

FIG. 1 shows a known C-arm angiography system having an industrial robot as a carrying device;

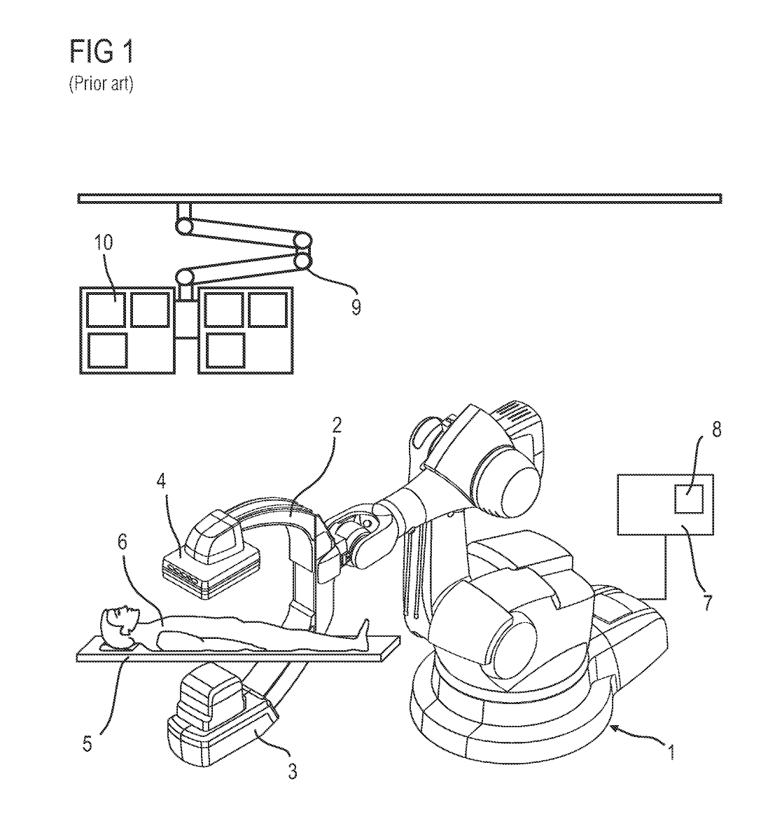

FIG. 2 shows a schematic illustration in explanation of known digital subtraction angiography; and

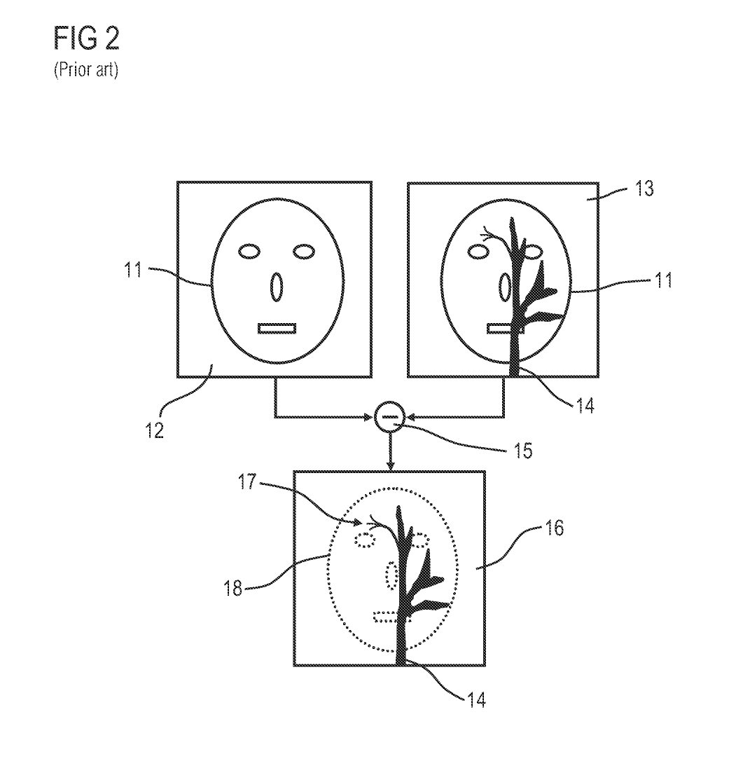

FIG. 3 shows a sequence of a process according to an embodiment for visualization only of regions of interest.

### DETAILED DESCRIPTION

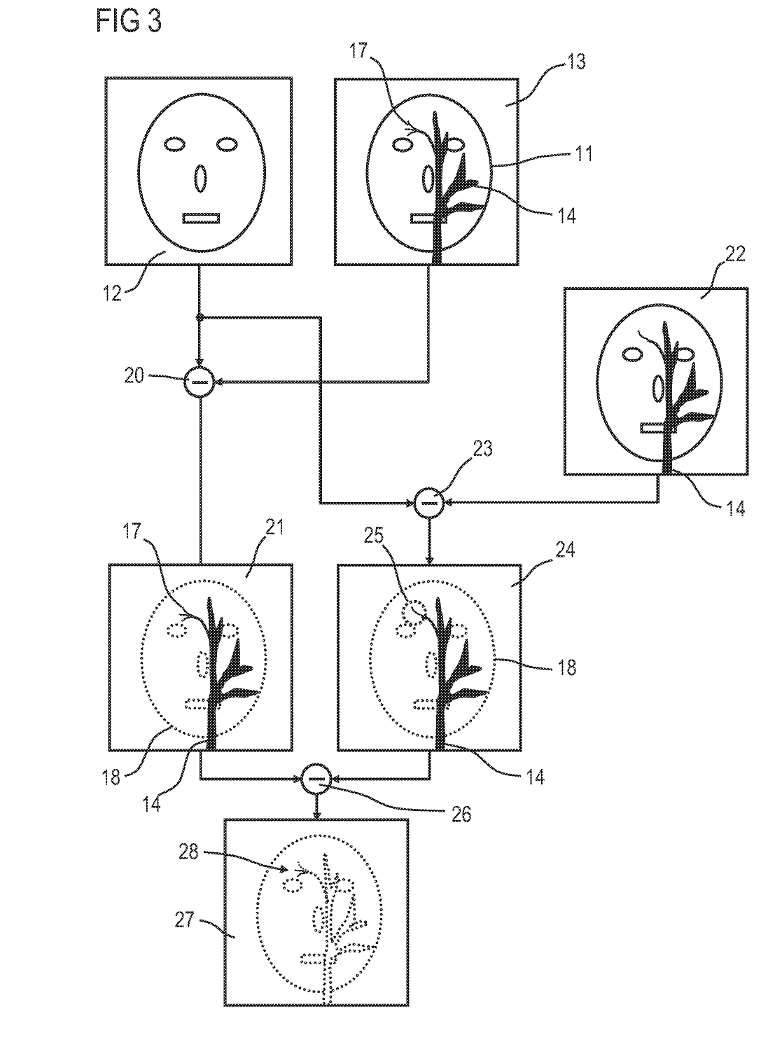

A DSA method according to one or more of the present embodiments is illustrated in FIG. 3. A mask image 12 is created where applicable from a plurality of chronologically sequential X-ray images prior to an operation. A post-contrast image 13 with a vascular tree 14 is generated from, where applicable, a plurality of chronologically sequential X-ray images created in the filling phase.

In a pre-operative first subtraction 20, the mask image 12 and the post-contrast image 13 are subtracted from one another such that a pre-operative subtraction image 21, in which only the vascular tree 14 may be recognized, is obtained. In the event of imprecise registration, an outline 18 of the skull may, however, only be faintly recognized, as is indicated by the dotted reproduction thereof in the pre-operative subtraction image 21.

Following a carotid operation or carotid stenting, a post-operative post-contrast image 22 with the vascular tree 14 is acquired from, where applicable, a plurality of chronologically sequential X-ray images created in the filling phase.

The digital mask image 12 is subtracted in a post-operative subtraction 23 from the subsequent post-operative post-contrast images 22. What remains are only parts or regions of the image that exhibit differences (e.g., the blood vessels of the vascular tree 14 filled with contrast medium), as may be recognized in a post-operative subtraction image 24. In the post-operative subtraction image 24, there may be blocked regions 25 in which the fine branching 17 may at least partially no longer be recognized, caused by distal blocking of the flow of contrast medium due to embolisms. In the overall post-operative subtraction image 24, the blocked regions 25 may, however, only be distinguished with difficulty.

Therefore, according to one or more of the present embodiments, in a third subtraction 26, the pre-operative subtraction image 21 and the post-operative subtraction image 24 are subtracted from one another such that a third subtraction image 27, in which essentially only the blocked vessels 28 are still shown, results. The third subtraction image 27 is reproduced for assessment and diagnosis on the displays of the suspended monitor bracket 10 supported by the carrier system 9 such that a visual inspection may be performed by a physician or investigator in order to diagnose a possible stroke.

Instead of the pre-operative mask image 12 for performing the post-operative second subtraction 23, a post-operative mask image may be captured immediately prior to the flooding with contrast medium for the post-operative acquisition of the post-contrast images 22.

In the event of imprecise registration, in addition to the outline 18 of the skull, the outlines of the remaining vascular tree may, however, also be faintly recognized, as the dotted reproduction thereof in the third subtraction image 27 is intended to express.

The visualization of microembolizations proposed according to one or more of the present embodiments is carried out using a technique similar to that employed in digital subtraction angiography. As a result of performing a subtraction on two “similar” images, the common image portions that detract from the diagnosis or mask the diagnosis are removed in order to thereby emphasize the differences. In the case of the DSA, the differences are the vessels. In the case of the method presented here, the differences are the vascular differences caused by microembolizations.

For the method according to one or more of the present embodiments, (e.g., in the case of the DSA) a largely “stable” region of investigation is to be provided. The image differences are thus not, for example, caused by movement. This is the case, for example, when imaging the skull 11 or the periphery.

The method according to one or more of the present embodiments is based on producing two angiographs (e.g., with regard to a carotid operation or a carotid stenting). One angiograph is produced directly prior to the intervention (e.g., pre-operatively), and one angiograph is produced after the intervention (e.g., post-operatively).

Any hits that may have occurred may be recognized in the post-operative angiograph by distal blocking of the contrast medium flow in the marker circles of the blocked regions 25 (e.g., the vessels are no longer visible as a result of the blocking). If only small vessels are closed, these areas may, however, only be very small and thus visible with difficulty or easily overlooked.

A new image is generated by forming the difference of these two angiographs (e.g., angiographic difference). In this image, the common image regions that are thus irrelevant to the diagnosis are subtracted away, which provides that precisely only the blocked vessels 28 are visible, thereby accordingly facilitating the diagnosis.

The method described above may be applied reliably in the case of completely motionless objects. In order to also make the method practicable in the case of small movements and variations, such as occur even in neuroradiology, the method according to one or more of the present embodiments may also be expanded in the following fashion. In order to avoid variations due to differing contrast fills, the comparison may not be performed on two individual images of the DSA sequences but on summed images of an entire sequence (e.g., “maximum opacification image”). In order to prevent motion artifacts within the individual DSA sequences, a motion compensation may be performed during the DSA (e.g., compensation between mask image and post-contrast image by “flexible pixel shift”). In order to avoid motion artifacts of the two DSA sequences with respect to one another, a registration of the DSAs with one another may be performed prior to the subtraction. This may, for example, be a rigid or flexible 2D/2D registration in the case of DSA images or a corresponding 3D/3D registration in the case of volume data sets.

In another embodiment, the method may be expanded from 2D angiographs to 3D rotational angiographs, which provides that no DSAs are then subtracted from one another, but 3D visualizations of the vascular system. The method may also be expanded to other 3D volumes such as, for example, 3D blood-flow or 3D perfusion visualizations. The difference occurring may also be overlaid on or merged with one of the DSAs in color-coded fashion. In this case the “blocked” portions remaining in the subtraction image are then simply superimposed in color in one of the DSA or volume images or one of the DSA or volume sequences, such that although the “blocked” portions are clearly visible, the “blocked” portions do, however, remain in the anatomical context. The result may also be visualized in color-coded fashion, or two likewise color-coded angiographs may be subtracted from one another. In the 3D case, the result may also be visualized dynamically or in color-coded fashion (e.g., by a “4D DSA,” or two 4D DSA sequences may be subtracted from one another).

A color-coded visualization having color properties characteristic of a point in time of a sequence of X-ray images is described, for example, in U.S. Pat. No. 7,729,525 B2.

The method may be applied in all areas of medicine in which 2D or 3D angiographs may be produced (e.g., in applications in the periphery with regard to arms or legs or concerning the heart).

The DSA difference principle according to one or more of the present embodiments for 2D images may be summarized by the following description. In the case of a carotid operation or a carotid stenting, angiographs are captured immediately prior to and after the intervention. Any hits that may have occurred may be recognized in the angiograph by distal blocking of the contrast medium flow. The vessels are no longer visible in the post-operative post-contrast images 22 and post-operative subtraction images 24. As a result of forming the difference, an image in which the blocked regions 25 remain visible is produced, thereby accordingly facilitating the diagnosis.

Instead of forming the difference between 2D angiographs, according to one or more of the present embodiments, it is also possible to compare volume data from so-called rotational angiographs (3D DSAs) in accordance with the same principle.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An angiographic examination method for performing a subtraction angiography for detection of microembolizations using an X-ray emitter and an X-ray image detector mounted at ends of a C-arm, a patient support table having a table surface for supporting a patient, a system control unit, an imaging system, and a monitor, the angiographic examination method comprising:
capturing at least one pre-operative pre-contrast image and at least one pre-operative post-contrast image having a vascular tree filled with contrast medium;
producing a pre-operative subtraction image, the producing of the pre-operative subtraction image comprising subtracting the at least one pre-operative pre-contrast image and the at least one pre-operative post-contrast image;
capturing at least one post-operative post-contrast image having a vascular tree filled with contrast medium;
producing a post-operative subtraction image, the producing of the post-operative subtraction image comprising subtracting a pre-contrast image of the at least one pre-operative pre-contrast image and the at least one post-operative post-contrast image;
producing another subtraction image, the producing of the other subtraction image comprising subtracting the pre-operative subtraction image and the post-operative subtraction image; and
displaying the other subtraction image.

2. The angiographic examination method of claim 1, wherein capturing the at least one pre-operative pre-contrast image, capturing the at least one pre-operative post-contrast image, and capturing the at least one post-operative post-contrast image comprises adding images of an entire sequence together.

3. The angiographic examination method of claim 1, further comprising performing a motion compensation during at least one of the subtractions.

4. The angiographic examination method of claim 1, wherein the at least one pre-operative pre-contrast image, the at least one pre-operative post-contrast image, the pre-operative subtraction image, the post-operative subtraction image, and the other subtraction image are obtained from volume data from 3D rotational angiographs.

5. The angiographic examination of claim 4, wherein the method reproduces 3D blood-flow or 3D perfusion visualizations.

6. The angiographic examination method of claim 1, further comprising performing at least one registration prior to subtractions.

7. The angiographic examination method of claim 6, wherein the at least one registration is a rigid or flexible 2D/2D registration in the case of DSA images, or a 3D/3D registration in the case of volume data sets.

8. The angiographic examination method of claim 1, further comprising overlaying contents of the other subtraction image in color-coded fashion onto the post-operative subtraction image.

9. The angiographic examination method of claim 1, wherein the pre-operative subtraction image and the post-operative subtraction image are visualized in color-coded fashion and subtracted from one another in color-coded fashion.

10. The angiographic examination method of claim 1, wherein 4D DSA sequences are subtracted from one another in order to perform at least one of the subtractions.

11. The angiographic examination method of claim 2, further comprising performing a motion compensation during at least one of the subtractions.

12. The angiographic examination method of claim 11, wherein the at least one pre-operative pre-contrast image, the at least one pre-operative post-contrast image, the pre-operative subtraction image, the post-operative subtraction image, and the subtraction image are obtained from volume data from 3D rotational angiographs.

13. The angiographic examination of claim 12, wherein the method reproduces 3D blood-flow or 3D perfusion visualizations.

14. The angiographic examination method of claim 3, further comprising performing at least one registration prior to subtractions.

15. The angiographic examination method of claim 14, wherein the at least one registration is a rigid or flexible 2D/2D registration in the case of DSA images, or a 3D/3D registration in the case of volume data sets.

16. The angiographic examination method of claim 15, further comprising overlaying contents of the subtraction image in color-coded fashion onto the post-operative subtraction image.

17. The angiographic examination method of claim 7, wherein the pre-operative subtraction image and the post-operative subtraction image are visualized in color-coded fashion and subtracted from one another in color-coded fashion.

18. The angiographic examination method of claim 7, wherein 4D DSA sequences are subtracted from one another in order to perform at least one of the subtractions.
